(19) United States
(12) Patent Application Publication
(10) Pub. No.: US 2007/0042344 A1
(43) Pub. Date: Feb. 22, 2007

(54) Differentiation regulating agent containing gene which regulating differentiation from stem cells into natural killer cells as effective ingredient

(76) Inventors: Inpyo Choi, Taejeon-si (KR); Hyung-Sik Kang, Taejeon-shi (KR); Suk-Ran Yoon, Taejeon-si (KR); Eun-Mi Kim, Taejeon-si (KR)

Correspondence Address: JHK LAW, P.O. BOX 1078, LA CANADA, CA, 91012-1078, US

(21) Appl. No.: 10/597,305

(22) PCT Filed: Jan. 20, 2005
(86) PCT No.: PCT/KR05/00188

§ 371 Date: Jul. 19, 2006

(30) Foreign Application Priority Data
Jan. 20, 2004 (KR) 10-2004-0004308

(51) Int. Cl. C12Q 1/00; C12Q 1/68; C12N 5/08

(52) U.S. Cl. 435/4; 435/6; 435/455; 435/368

(57) ABSTRACT

The present invention relates to a cell differentiation regulating agent containing a gene regulating differentiation from stem cells into natural killer cells as an effective ingredient, more precisely, a cell differentiation regulating agent containing a gene regulating differentiation from stem cells into premature natural killer cells as an effective ingredient and a screening method of the gene by taking advantage of SAGE. The gene of the present invention is a novel one that is confirmed not to be like any other known genes regulating differentiation from stem cells into natural killer cells. Though, the gene can be easily screened by SAGE and a natural killer cell differentiation-regulating agent containing the gene as an effective ingredient can be effectively used as an anticancer agent.

### TECHNICAL FIELD

The present invention relates to a differentiation-regulating agent containing a gene regulating differentiation from stem cells into natural killer cells as an effective ingredient and a screening method for the gene.

### BACKGROUND ART

Stem cells have multipotency for the differentiation into various organs and have self-renewal capacity, and are found in both embryos and adults. The stem sells enable differentiation of a cell into a specific cell or an organ, so that our attention has been focused on the possibility to use the stem cells for organ transplantation or cell therapy.

Hematopoietic stem cells, a kind of adult stem cells, are the cells that can be differentiated into every blood forming cells such as erythrocytes, leucocytes, platelets and lymphocytes. And cells involved in immune system are continuously self-renewed from the hematopoietic stem cells in bone marrow. Hematopoietic stem cells have been used so far for the treatment of various blood diseases including cancer by means of bone marrow transplantation. According to recent reports, the hematopoietic stem cells could be differentiated into other types of cells such as muscle, nerve, bone, etc, in animal models. If they can be applied to human, the hematopoietic stem cells can be used for the treatment of in variety of diseases including diabetes, Parkinson's disease, spinal cord injury, etc, because they can replace other cells and organs successfully.

In particular, natural killer (referred as ‘NK’ hereinafter) cells destroy cancer cells non-specifically. Owing to their cytotoxic capacity, NK cells have now been in use for the treatment of a solid tumor using LAK (lymphokine activated killer cell) and TIL (tumor infiltration lymphocytes) and for immune therapy (J Immunol., 1986, 36(10):3910-3915; Hematologia, 1999, 84:1110-1149) using donor lymphocyte infusion, suggesting that it further makes the way to new cell therapy to reduce rejection after bone marrow transplantation or organ transplantation. It was also reported that the defect in differentiation and activation of NK cells is related to various diseases including breast cancer (Breast Cancer Res Treat., 2003, 66(3):255-263), melanoma (Melanoma Res., 2003, 13(4):349-356) and lung cancer (Lung Cancer, 2002, 35(1):23-18), so that NK cell therapy draws our attention to treat such diseases.

Thus, the present inventors have identified a novel gene regulating differentiation of stem cells into NK cells by using SAGE (Serial Analysis of Gene Expression) and have completed this invention by confirming that NK cell differentiation is regulated by the gene above and further the gene can be a great aid for the treatment of diseases including cancer.

[Disclosure]

[Technical Problem]

It is an object of the present invention to provide a NK cell differentiation-regulating agent containing a gene which regulating differentiation from stem cells into natural killer cells as an effective ingredient and a screening method for the gene using SAGE.

[Technical Solution]

In order to achieve the above object, the present invention provides a differentiation-regulating agent which regulates differentiation from stem cells into natural killer cells.

The present invention also provides a differentiation-regulating agent which regulates differentiation from stem cells into premature natural killer cells.

The present invention further provides a differentiation-regulating agent which regulates differentiation from premature natural killer cells into mature natural killer cells.

The present invention also provides an anticancer agent developed by using the differentiation-regulating agent of the invention.

The present invention further provides a screening method for a gene regulating differentiation from stem cells into natural killer cells, based on SAGE.

In the present invention, ‘differentiation regulating gene’ means every gene that regulate differentiation from stem cells into natural killer cells, that is, they can accelerate or inhibit differentiation. More precisely, differentiation-regulating gene of the present invention can accelerate differentiation, so that it promotes a progress to the next stage. In the meantime, it also has functions of maintaining each stage or inhibiting a progress to the next stage.

In the present invention, ‘SAGE’ stands for ‘serial analysis of gene expression’. SAGE can be performed either by conventional method or by manufacturer's protocol (Invitrogen™ life technologies) (http://www.invitrogen.com).

The mark in bracket after the name of gene means GenBank ID implying sequence of each gene and the GenBank ID can be easily searched and used by the people in this field.

Type II restriction enzyme used in the present invention is a conventional enzyme widely used in the field of genetic engineering. It needs magnesium ions to activate and recognizes a specific nucleotide sequence of DNA, so that it can cut exactly the wanting area or the neighboring area apart from the recognized nucleotide sequence. Type II S restriction enzyme used in the present invention means NlaIII (recognizes and digests the area of CATG region every 250 base pairs).

Hereinafter, the present invention is described in detail.

The present invention provides a differentiation regulating agent for natural killer cells which is characterized by containing one or more genes, as an effective ingredient, selected from a group consisting of homeobox protein MIX (AF15457), pre-pro-proteinase 3 (U97073), myeloblastosis (Myb) oncogene (M16499), keratin complex 1, acidic, gene 13 (NM_{—}010662), PA-phosphatase related phosphoesterase (AK002966), gamma-parvin (BC011200), forkhead-related transcription factor 1C (AF330105), RIKEN cDNA 5730501N20 gene (AK017744), c-myc protein (X010223), ribosomal protein L10A (AK002613), Oct 2b gene (X53654), microlite (AK015601), dihydrolipoamide dihydrogenase (BC003368), tracle (U81030), lysozyme (BC002069), ferritin H chain (BC012314), brevican (X87096), matrix metalloproteinase 12 (BC019135), EIA-stimulated gene cellular inhibitor (AF084524), S100 calcium binding protein A9 (BC027635), MPS1 protein (L20315), transglutaminase 2 (BC016492), serum and glucocorticoid regulated protein kinase (AF139639), RIKEN cDNA 5830413L19 (BC027496), interferon-induced protein (BC003804), milk fat globule membrane protein EGF factor 8 (BC018577), cell-surface glycoprotein p91 (U83172), arginase 1 (BC050005), tumor necrosis factor receptor 1 (M59378), retinoid-induced serine carboxypeptidase (AF330052), FLJ11000 homologue (BC023802), interleukin-18 binding protein d precursor (AF110803), chloride channel 7 (AK009435), CD36 antigen (BC010262), zink finger protein homologue (BC030186), carbohydrate binding protein 35 (J03723), C-type calcium dependent carbohydrate (BC003218), lipoprotein lipase (NM_{—}008509), v-maf lacertus fibrosarcoma oncogene (BC038256), interleukin 7 receptor (NM_{—}008372), chemokine (C-C) receptor 1 (BC011092), neurophilline (MGD|MGI:106206) (AK002673), SERPINA3G (XM_{—}127137), GABA-A receptor subunit 6 (X51986), LAPTm5 (U51239), G-protein signal regulator (BC049968), decoy-stimulating factor GPI fixed mRNA (L41366), Y box protein 3 (AK019465), osteopontin precursor (J04806), amyloid beta (A4) precursor protein-binding family (AK021331), T cell receptor beta subunit analogue (U63547), immune related nucleotide 1 (BC005577), higher stage transcription factor 1 (NM_{—}009480), olfactory receptor MOR267-7 (NM_{—}146714), lymphocyte specific protein tyrosine kinase (M12056), osteoclast cancer inhibitor (AB013898), platelet active receptor homologue (BC024054), natural killer cell protein 2-A1 (AF016008), unidentified protein MGC36662 (BC023851), semaphorin 6A precursor homologue (AK004390), neurofilament homologue polypeptide (BC025872), cornin homologue actin binding protein 2A (BC026634), solute transmitting family 6 (BC015245), temporary purine receptor P2Y10 homologue (AK020001), T cell receptor gamma chain (X03802), poly A polymerase alpha (NM_{—}011112), OPA-related protein OIP5 analogue (AK017825) and mytogen activated protein kinase 1 analogue (BC006708).

The present invention also provides a differentiation regulating agent which regulates differentiation from stem cells into premature natural killer cells which is characterized by containing one or more genes selected from a group consisting of homeobox protein MIX (AF15457), pre-pro-proteinase 3 (U97073), myeloblastosis (Myb) oncogene (M16499), keratin complex 1, acidic, gene 13 (NM_{—}010662), PA-phosphatase related phosphoesterase (AK002966), gamma-parvin (BC011200), forkhead-related transcription factor 1C (AF330105), RIKEN cDNA 5730501N20 gene (AK017744), c-myc protein (X010223), ribosomal protein L10A (AK002613), Oct 2b gene (X53654), microlite (AK015601), dihydrolipoamide dihydrogenase (BC003368) and tracle (U81030), as an effective ingredient.

The present invention further provides a differentiation regulating agent which regulates differentiation from premature natural killer cells into mature natural killer cells which is characterized by containing one or more genes, as an effective ingredient, selected from a group consisting of lysozyme (BC002069), ferritin H chain (BC012314), brevican (X87096), matrix metalloproteinase 12 (BC019135), EIA-stimulated gene cellular inhibitor (AF084524), S100 calcium binding protein A9 (BC027635), MPS1 protein (L20315), transglutaminase 2 (BC016492), serum and glucocorticoid regulated protein kinase (AF139639), RIKEN cDNA 5830413L19 (BC027496), interferon-induced protein (BC003804), milk fat globule membrane protein EGF factor 8 (BC018577), cell-surface glycoprotein p91 (U83172), arginase 1 (BC050005), tumor necrosis factor receptor 1 (M59378), retinoid-induced serine carboxypeptidase (AF330052), FLJ11000 homologue (BC023802), interleukin-18 binding protein d precursor (AF110803), chloride channel 7 (AK009435), CD36 antigen (BC010262), zink finger protein homologue (BC030186), carbohydrate binding protein 35 (J03723), C-type calcium dependent carbohydrate (BC003218), lipoprotein lipase (NM_{—}008509), v-maf lacertus fibrosarcoma oncogene (BC038256), interleukin 7 receptor (NM_{—}008372), chemokine (C-C) receptor 1 (BC011092) and neurophilline (MGD|MGI:106206).

The present invention also provides a differentiation regulating agent which regulates differentiation of mature natural killer cells which is characterized by containing one or more genes, as an effective ingredient, selected from a group consisting of SERPINA3G (XM_{—}127137), GABA-A receptor subunit 6 (X51986), LAPTm5 (U51239), G-protein signal regulator (BC049968), decoy-stimulating factor GPI fixed mRNA (L41366), Y box protein 3 (AK019465), osteopontin precursor (J04806), amyloid beta (A4) precursor protein-binding family (AK021331), T cell receptor beta subunit analogue (U63547), immune related nucleotide 1 (BC005577), higher stage transcription factor 1 (NM_{—}009480), olfactory receptor MOR267-7 (NM_{—}146714), lymphocyte specific protein tyrosine kinase (M12056), osteoclast cancer inhibitor (AB013898), platelet active receptor homologue (BC024054), natural killer cell protein 2-A1 (AF016008), unidentified protein MGC36662 (BC023851), semaphorin 6A precursor homologue (AK004390), neurofilament homologue polypeptide (BC025872), cornin homologue actin binding protein 2A (BC026634), solute transmitting family 6 (BC015245), temporary purine receptor P2Y10 homologue (AK020001), T cell receptor gamma chain (X03802), poly A polymerase alpha (NM_{—}011112), OPA-related protein OIP5 analogue (AK017825) and mytogen activated protein kinase 1 analogue (BC006708).

A gene included in the differentiation regulating agent of the present invention has functions of 1) regulating differentiation from stem cells into premature NK cells, 2) regulating differentiation from premature NK cells into mature NK cells, and 3) regulating differentiation of mature NK cells, and a differentiation regulating gene functioning at each stage can be independently used as a differentiation regulating agent from stem cells into NK cells. In the preferred embodiment of the present invention, differentiations from stem cells into premature NK cells and into mature NK cells were induced by culturing HSC cells with the treatment of cytokine (FIG. 1a-FIG. 1c). From each stage, a whole RNA was separated and SAGE was performed as shown in a schematic diagram of FIG. 2. By SAGE, genes showing a specific increase of expression were selected from each differentiation stages (FIG. 3a-FIG. 3f). The genes were compared with others deposited at GenBank. As a result, the genes were none of those reported to have functions of regulating differentiations from stem cells into pNK cells (see Table 3), from pNK cells into mNK cells (see Table 4) and of mNK cells (see Table 5).

Therefore, the genes of the present invention are a new founding having a novel differentiation regulating mechanism, and a pharmaceutical composition having one or more of those genes can be used for the regulation of cell differentiation. In particular, a differentiation regulating agent involved in the differentiation from stem cells into premature NK cells can be prepared by using one or more of genes listed in Table 3, and also a differentiation regulating agent involved in the differentiation from premature NK cells into mature NK cells can be prepared by using one or more genes listed in Table 4. A differentiation-regulating agent involved in the differentiation of mature NK cells can be prepared by using one or more genes listed in Table 5. All the genes listed in Table 3, 4 and 5 have functions of regulating the differentiation from stem cells into NK cells, so a differentiation regulating agent which regulates differentiation of natural killer cells can be prepared by using one or more genes mentioned above.

Cell differentiation regulating agent of the present invention can also be used for the treatment of cancers. The differentiation-regulating agent of the invention is preferably applicable to such cancers as breast cancer, melanoma and lung cancer. The defects of NK cell differentiation and activation result in various cancers, for example, breast cancer (Breast Cancer Res Treat., 2003, 66(3) :255-263), melanoma (Melanoma Res., 2003, 13(4) :349-356) and lung cancer (Lung Cancer, 2002, 35(1):23-18). Thus, the mentioned cancers can be effectively treated by regulating NK cell differentiation with the NK cell differentiation-regulating agent of the present invention.

The cell differentiation-regulating agent of the present invention can be administered orally or parenterally and be used in general forms of pharmaceutical formulation. The cell differentiation-regulating agent of the present invention can be prepared for oral or parenteral administration by mixing with generally used fillers, extenders, binders, wetting agents, disintegrating agents, diluents such as surfactant, or excipients. Solid formulations for oral administration are tablets, pill, dusting powders, granules and capsules. These solid formulations are prepared by mixing with one or more suitable excipients such as starch, calcium carbonate, sucrose or lactose, gelatin, etc. Except for the simple excipients, lubricants, for example magnesium stearate, talc, etc, can be used. Liquid formulations for oral administrations are suspensions, solutions, emulsions and syrups, and the abovementioned formulations can contain various excipients such as wetting agents, sweeteners, aromatics and preservatives in addition to generally used simple diluents such as water and liquid paraffin. Formulations for parenteral administration are sterilized aqueous solutions, water-insoluble excipients, suspensions, emulsions, and suppositories. Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerol, gelatin, etc.

The effective dosage of the agent of the present invention is 0.1˜0.2 mg/kg, and preferably 0.15 mg/kg. The administration times of the agent of the present invention might be once to three times a day.

The present invention also provides a screening method for a gene regulating the differentiation from stem cells into natural killer cells comprising the following steps:

1) Synthesizing cDNA after separating whole RNA from cells;

2) Separating tag after digesting the cDNA of the step 1;

3) Connecting each tag separated in the step 2 and then analyzing nucleotide sequence thereof; and

4) Quantifying the expression of the gene, based on the analyzed nucleotide sequence above, by using SAGE analyzing program.

In the step 1, cells are preferably selected from each stage of differentiation from stem cells into natural killer cells. In the preferred embodiment of the present invention, hematopoietic stem cells (HSC) were used as stem cells, and premature natural killer cells and mature natural killer cells were used as natural killer cells. Any conventional method, if only it is able to separate whole RNA from sample with high yield and with preventing RNase contamination, can be used (Sambrook, et al., 1989, Molecular Cloning). In general, it is easy to follow manufacturer's protocol to separate RNA by using a RNA separating agent. In order to synthesize cDNA from a whole RNA, oligo dT primer was attached to a whole RNA, but that was not the only way to synthesize cDNA and any other method to synthesize cDNA could be used. In the preferred embodiment of the present invention, oligo dT primer was attached to a whole RNA to synthesize cDNA and at that time, oligo dT primer was to insert poly A sequence for the synthesis of mRNA. It is preferred that 20-30 T sequences are repeated in oligo dT primer. And, it is also allowed that magnetic beads are additionally attached to one end of the oligo dT primer, because tag can be successfully separated without contamination by using magnetic beads.

In the step 2, the process of separating tag after digesting the cDNA is composed of the following steps:

a) Preparing tag by digesting cDNA with IIS type restriction enzyme 1;

b) Combining two kinds of adapters each including a US type restriction enzyme 1 recognition site at one end cleavage site of the tag prepared in the step a;

c) Separating tag by digesting the tag connected to the adapter in the step b with IIS type restriction enzyme 2 and cutting off oligo dT magnetic beads from the tag;

d) Preparing ditag by combining the tags prepared in the step c each other; and

e) Preparing ditag only by digesting the ditag prepared in the step d with IIS type restriction enzyme 1 and cutting off the adapter.

In the step a, the reason why the synthesized cDNA was digested with IIS type restriction enzyme 1 was that the cleavage site digested by the enzyme could be prepared as a tag binding site and in fact it was easy to use the area for binding with tag because the cleavage site formed 5′ overhangs. As a IIS type restriction enzyme 1, any adequate enzyme is possible and NlaIII restriction enzyme is preferred. That is because cDNA has NlaIII restriction enzyme recognition sites at every 250 bp, so that regular sized tag can be easily prepared by digesting cDNA with the enzyme.

In the step b, two kinds of adapters to be linked to the cleavage site of tag have about 40 bp long sequences that are bound each other complementarily. The adapters include NlaIII restriction enzyme recognition site (CATG) at one end, to which tag is bound, and form overhangs which make the bond with tag easy.

In the step c, the tag bound to the adapter was digested with IIS type restriction enzyme 2. IIS type restriction enzyme 2 was bound to the restriction enzyme site of an adapter to cut the area located at 10-14 bp downstream from the restriction enzyme cleavage site, resulting in the separation of about 50 bp long tag containing the end of 4 bp size overhang at 5′ end. BsmFI was preferably used as a IIS type restriction enzyme 2.

In the step d, tags were connected each other to form a ditag. Precisely, the end of overhang was formed at each 5′ end of the tags, so that a ditag could be easily formed by connecting those ends. The resultant ditag was about 100 pb long.

In the step e, the ditag was digested with IIS type restriction enzyme 1 to cut the adapter off, resulting in pure ditag only. Precisely, the binding area where the end of tag and an adapter were bound included IIS type restriction enzyme recognition site, so the adapter could be cut off by using the IIS type restriction enzyme 1. As a result, about 26 bp long pure ditag was prepared.

In the mean time, in the step 3, 10 to 20 tag fragments, obtained in the step 2, were bound and their nucleotide sequences were investigated. And the investigation process was composed of the following steps:

a) Cloning the concatemer type ditag prepared by binding ditags prepared in step 2 into a vector; and

b) Investigating nucleotide sequence of tag of the vector used for cloning in step a.

In the step a, ditags were bound to form a concatemer. Precisely, both ends of a ditag included US type restriction enzyme 1 recognition site, indicating that overhang could be formed. Such ditags could be connected easily and so about 20 to 50 tags were connected to form a concatemer. The prepared concatemer type tag was inserted into a conventional vector for cloning to investigate nucleotide sequence thereof. In the preferred embodiment of the present invention, pZerO-1 vector was used for the cloning. The mentioned expression vector was included in a kit (Invitrogen Life Science) provided for SAGE analysis and was very useful.

In the step 4, the expression was quantified by investigating nucleotide sequence obtained above with SAGE analyzing program. Precisely, the obtained nucleotide sequence was compared with other sequences of genes deposited at GenBank to identify it. Then, SAGE analyzing program was used to classify sequences from ones with high expression to others with low expression. They were marked with red, yellow, green and blue after clustering, making the expression levels be shown clearly. And the amount of expression can be evaluated as a numerical value. SAGE analyzing program can be either provided by a company or one of soft wares provided through internet. In the present invention, a conventional program (cluster and treeview computer program, http://rana.1b1.gov) widely used for clustering of SAGE results was used.

A screening method of the present invention is based on SAGE analysis. Each step of the method was performed by taking advantage of the general SAGE analysis or could be performed by modified processes according to manufacturer' instruction. The outline of the method of the invention is shown in a schematic diagram of FIG. 2.

### DESCRIPTION OF DRAWINGS

FIG. 1a-FIG. 1c show the comparison of expressions of surface molecules during the differentiation processes from mouse hematopoietic stem cells (HSC) through premature NK cells (pNK) to mature NK cells (mNK) in the presence (+OP9) or in the absence (−OP9) of OP9 interstitial cells.

FIG. 1a is a set of graphs showing the purity of cells of each stage of NK cell differentiation which was presented by two different colors determined by flow cytometry. The numbers of each quadrant indicate percentage of corresponding cells.

Lin− c-kit+: (96%), CD122+ NK1.1−: (95%),

CD122+ NK1.1+: (94%, 95% respectively)

FIG. 1b is a set of graphs showing the expressions of NK cell related surface markers (NK1.1, DX5, CD94, NKG2A) induced during the differentiation from premature NK cells into mature NK cells, for which OP9 interstitial cells were added for the culture.

FIG. 1c is a set of photographs showing the results of RT-PCR. Whole cytoplasmic RNA was extracted from cells from each stage of NK cell differentiation to investigate whether or not CD122, a representative NK cell related gene, and perforin were expressed.

FIG. 2 is a schematic diagram showing the SAGE process to detect a differentiation-regulating gene of the present invention.

FIG. 3a-FIG. 3f show clustering of gene expression profile obtained during NK cell differentiation by using SAGE analysis.

FIG. 3a shows the group of genes expressed most in HSC cells, FIG. 3b presents the group of genes expressed most in pNK cells, FIG. 3c shows the group of genes expressed most in mNK (−OP9) cells, and FIG. 3d presents the group of genes expressed most in mNK (+OP9) cells.

FIG. 3e shows genes inhibiting the activation of NK cells, and FIG. 3f shows genes promoting the activation of NK cells.

In the above FIG. 3a-FIG. 3f, from the clustering based on SAGE analysis, when the cluster frequency was over 80, it was marked red, when the frequency was 50-79, it was marked yellow, when the frequency was 30-49, it was marked green and when the frequency was under 29, it was marked blue.

FIG. 4a-FIG. 4d show the results of RT-PCR to investigate whether the gene that was confirmed by SAGE to regulate the differentiation of NK cells was actually expressed. The expression was quantified in comparison with the comparative beta-actin gene.

FIG. 4a shows genes expressed specifically in HSC cells during the NK cell differentiation, FIG. 4b presents genes expressed specifically in pNK cells, FIG. 4c shows genes expressed specifically in mNK cells, and FIG. 4d shows that LPL was treated to NK cells at different concentrations (250 ng/ml and 500 ng/ml) to investigate the effect of LPL on the differentiation of NK cells, and as a result, the differentiation into mNK cells was promoted.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Stem Cell Isolation From Bone Marrow

All bones including tibia and femur of a C57BL/6 mouse (Dae Han Biolink) at the age of 6-9 weeks were pulverized. The pulverized pieces were passed through 70-micron cell strainer and erythrocytes in them were removed by treating lysis solution (Sigma, St. Louse, Mo.) to obtain bone marrow cells only. The bone marrow cells were reacted with antibody markers that were biotin labeled for systemic markers (CD11b: macrophage marker, Gr-1: granulocyte marker, B220: B cell marker, NK1.1: NK cell marker, CD2: T cell marker, TER-119: erythrocyte marker), followed by washing. Then, the cells were reacted with streptavidin labeled magnetic beads (Miltenyi Biotec, Auburn, Calif.). Magnetic labeled Lin+ cells were scavenged by being passed through CS column (Miltenyi Biotec) in the magnetic field of MACS (Miltenyi Biotec). The remaining Lin− cells passed through the column were reacted with magnetic beads connected to c-kit and then passed through MACS column (Miltenyi Biotec), resulting in c-kit+ cells remaining in the column. The purity of the obtained Lin− c-kit+ hematopoietic stem cells (referred as ‘HSC cells’ hereinafter) was measured by FACS (BD Bioscience, Mountainview, Calif.). As a result, it was confirmed that the cells had over 96% purity.

### EXAMPLE 2

### Inducement of the Differentiation From Stem Cells into NK Cells

HSC cells, separated from bone marrow in the Example 1, in RPMI complete medium supplemented with mouse SCF (30 ng/ml, BioSource, Camarillo, Calif.), mouse Flt3L (50 ng/ml, PeproTech, Rocky Hill, N.J.), mouse IL-7 (0.5 ng/ml, PeproTech), indomethacin (2 μg/ml, Sigma), gentamycin (20 μg/ml) and 10% fetal bovine serum were inoculated to a 6-well plate (Falcon) at the concentration of 2×10⁶ cells/well. The cells were cultured in a 37° C., 5% CO₂ incubator for 6 days. After 3 days from the culture, half of the supernatant was discarded and a fresh medium supplemented with cytokine along with the same composition as the above was added. 6 days later, CD122+ premature NK cells (referred as ‘pNK cells’ hereinafter) were separated with MACS using FITC labeled CD122 antibody and magnetic beads conjugated anti-FITC antibody. The purity of the premature NK cells was measured by FACS, and from the result, it was confirmed that the cells had over 92% purity.

In order to induce the differentiation into mature NK cells (referred as ‘mNK cells’ hereinafter), HSC cells were recovered after 6 days from the culture, and then cultured them only or with OP9 stromal cells (Science 1994, 265(5175): 1098-1101; Nakano T, Kodama H, Honjo T.: Generation of lymphohematopoietic cells from embryonic stem cells in culture) in the presence of mouse IL-15 (20 ng/ml, PeproTech). 3 days later, half of the medium was replaced with a fresh one having the same composition. On day 12, NK1.1+ cells were separated by using FITC labeled anti-NK1.1 antibody and magnetic beads conjugated anti-FITC antibody. Mature NK cells were investigated with flow cytometry using anti-CD122, NK1.1, DX5 and NK cell receptor antibodies.

### EXAMPLE 3

### Investigation of Purified NK Cell Phenotype Specific to the Differentiation Stages

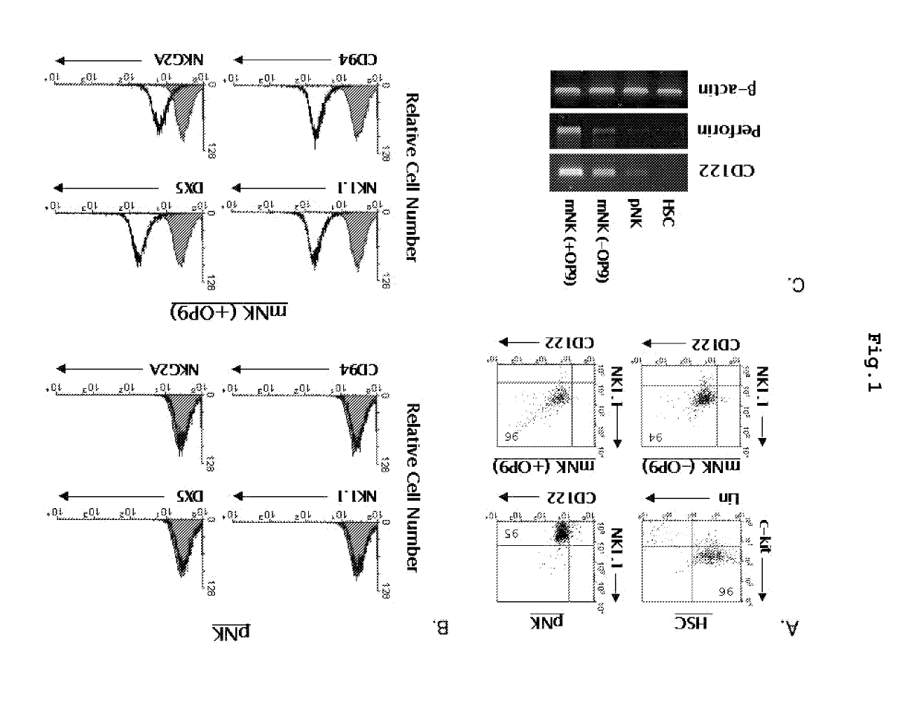

In order to collect specific NK cells from each differentiation stages, Lin− c-kit+ HSC (>95%) cells, separated from mouse bone marrow, were cultured in the presence of SCF, Fit-3L and IL-7 for 6 days. Then, CD122+ pNK cells were separated and analyzed by flow cytometry. In the case of mNK cells (−OP9 or +OP9), IL-15 cells were cultured only or with OP9 stromal cells for 6 more days. The recovered cells were analyzed by flow cytometry (FIG. 1a). When the cells were cultured together with OP9 stromal cells, the number of mNK cell was increased (−OP9; 94% and +OP9; >95%). Ly49 receptors on the surface of mNK cells play an important role in mNK cell functions and their expression is regulated by a signal transduction by the communication with other immune cells. In order to confirm whether or not the co-culture of HSC cells derived from bone marrow and stromal cells was essential for the expression of Ly49 receptors of mNK cells, mNK cells were cultured only or together with OP9 cells in the presence of IL-15 and then the expression of Ly49 was investigated (FIG. 1b). When the cells were cultured together with OP9 cells (+OP9), Ly49C/I and Ly49G2 were expressed in mNK cells. On the other hand, when the cells were cultured independently (−OP9), neither Ly49C/I nor Ly49G2 were expressed. The results indicate that the co-culture of HSC cells and OP9 cells is essential for the maturation of NK cells. After investigating the expressions of CD122 and perforin genes according to the differentiation stages of NK cells, HSC cells were proved to become mature to NK cells during the differentiation (FIG. 1c).

### EXAMPLE 3

### SAGE(Serial Analysis of Gene Expression)

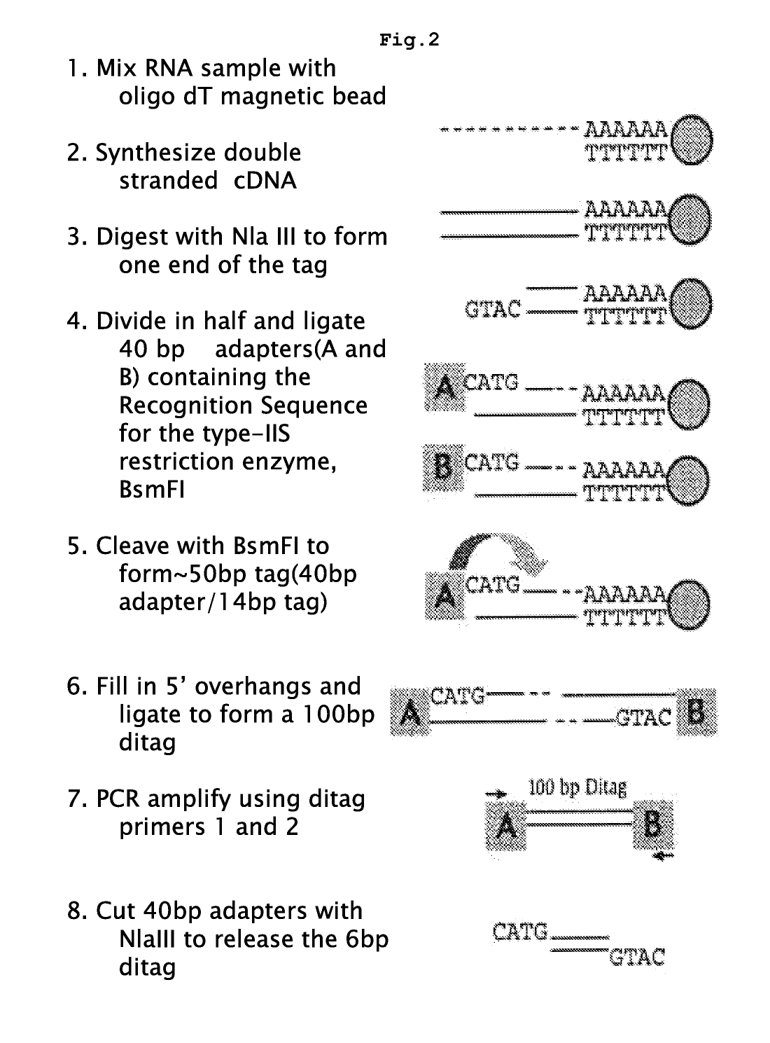

Whole RNA was extracted from HSC cells prepared in the Example 2 and from NK differentiation stage specific cells (pNK and mNK). mRNA was separated and purified from 5 μg of the whole RNA by using (dT)25 magnetic beads (Dynal A. S., Oslo, Norway). The mRNA, separated and purified by the oligo dT beads, was used as a template for the synthesis of cDNA by cDNA synthesis kit (Invitrogen, Life Technologies) using oligo (dT) primer that was 5′-biotinized and 3′-linked. According to the manufacturer's instructions (Invitrogen, Life Technologies), tag for SAGE was prepared from the cDNA by the method explained in the schematic diagram of FIG. 2. The cDNA was digested with restriction enzyme NIaIII and 3′-region was bound to magnetic beads (Dynal) coated with streptavidin. The tag was divided into two fractions, which were bound to linkers (Invitrogen, Life Technologies) having NIaIII recognition site, respectively. Linker binding tag was digested with BsmFI. The isolated tag and the linker were treated with Pfu DNA polymerase to make blunt-end. The blunt-ends were linked together to form a ditag. PCR was performed to amplify the ditag by using biotin labeled SAGE primer (Invitrogen, Life Technologies). Then, the ditag was digested with NIaIII to be separate from linker. T4 DNA ligase was treated thereto to form a concatemer. The prepared concatemer was cloned into Sph I pre-digested pZero-1 vector (Invitrogen, Carlsbad, Calif.) (FIG. 2). Then, the cloning product was amplified by PCR using M13 forward primer represented by SEQ. ID. No. 1 and M13 backward primer represented by SEQ. ID. No. 2. Amplified positive colony was collected, and then the sequence was investigated by sequencing kit (Big-Dye sequencing kit) and nucleotide sequencer (ABI377 sequencer, Perkin-Elmer Applied Biosystems, Branchburg, N.J.). The sequence of tag was identified by SAGE 300 soft ware.

### EXAMPLE 4

### SAGE Data Analysis

<4-1> Bioinformatical Analysis

Reference SAGE-tag database was established from UniGene mouse database harboring most sequences expressed in a mouse, which was filed in GenBank. SAGE tag was determined by (i) direction of each transcript, (ii) presence or absence of poly(A) signal (AATAAA or ATTAAA), (iii) presence or absence of poly A tail, and (iv) presence or absence of the last CATG cleavage site in a sequence. All SAGE tags extracted from reference sequences were used for the construction of reference SAGE database. Experimental SAGE tag was matched with reference SAGE database (http://www.hpcl.cs.uchicago.edu/gist). In order to identify a gene corresponding to each SAGE tag, a computer program SAGEmap (Lash A. E et al., 2000) was used.

<4-2> Analysis of Clustering According to Quantitative Distribution of SAGE Profile

A clustering computer program (cluster and treeview computer program, http://rana.1b1.gov) was used to investigate clustering of SAGE data obtained in the Example 4-1, based on other expressions and functional patterns shown during NK cell differentiation processes. Briefly, in each stage, different colors such as blue, green, yellow and red were marked according to the frequency (PERL script available upon request). Mid-point was included in the corresponding RGB value. According to the colorful results, some tags showing clear and high expression were selected and let them apart from each other in panel. The remaining tags were re-arranged, placing lines showing similar expression patterns beside in order to make gradual color change as a whole.

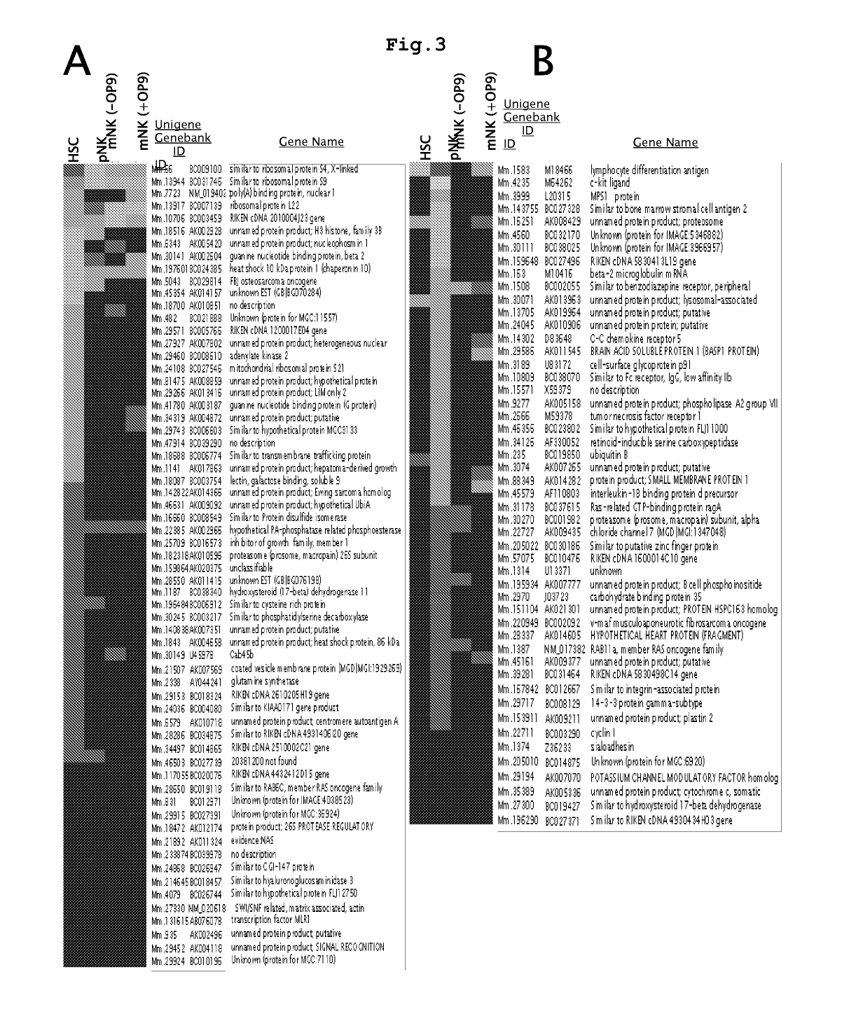

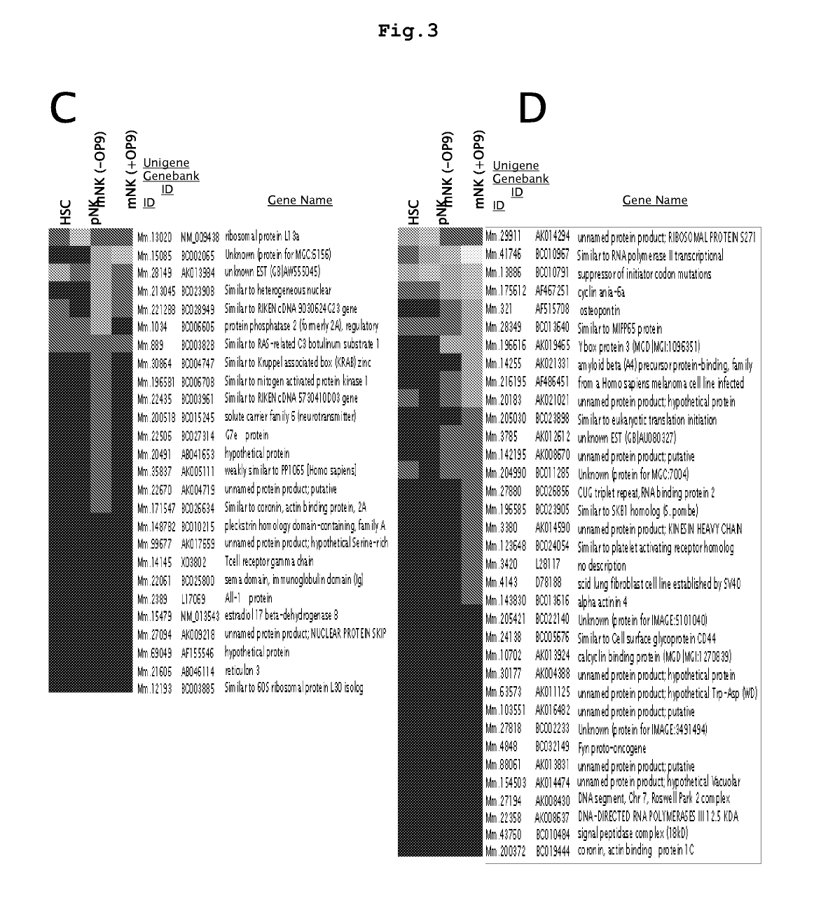

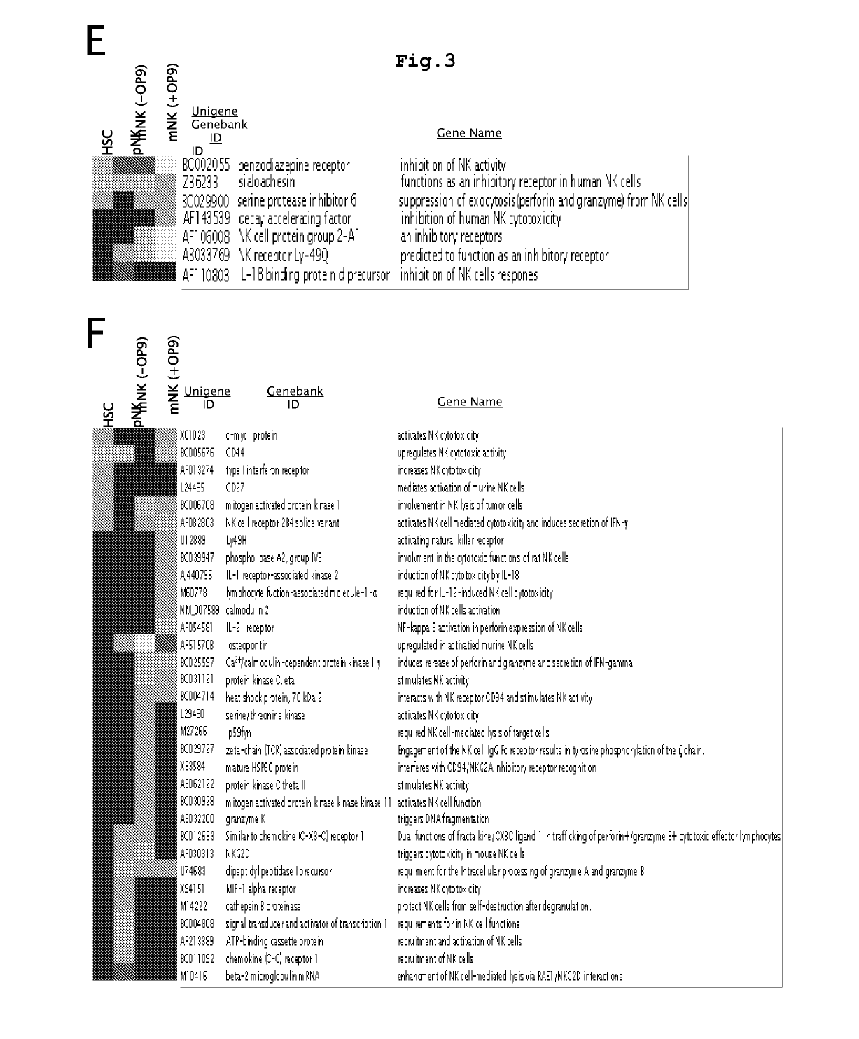

The increase or the decrease of gene expression during NK cell differentiation was investigated based on SAGE profiles of HSC, pNK, mNK(−OP) and mNK(+OP9) cells. As a result, as shown in FIG. 3a-FIG. 3f, the target genes were clustered into 4 groups. Precisely, FIG. 3a presents a gene group whose expression was increased in HSC but decreased by the NK cell differentiation, FIG. 3b shows a gene group whose expression is high in pNK cells and FIG. 3c presents a gene group whose expression was high in mNK(−OP9). FIG. 3d shows a gene group whose expression was gradually increased until it reached maximum in mNK(+OP9) cells. In particular, the gene group (FIG. 3b) showing the best expression in pNK cells includes many immune regulating genes such as lymphocyte differentiation antibody, C-C chemokine receptor, tumor necrosis factor and interleukin-18 binding protein, etc, indicating that immune regulating factors play an important role in pNK cell differentiation. Next, based on the informed database, genes were classified by the function of regulating the NK cell activity. FIG. 3e and FIG. 3f show genes inhibiting and promoting the NK cell activity, respectively. In most cases, those genes are expressed in late stage of differentiation. Genes involved in the cell activation include many signal factors such as mitogen activated protein kinase, phospholipase A2, IL-2 receptor, chemokine receptor, etc.

### EXAMPLE 5

### Analysis of Genes Regulating Each Stage of NK Cell Differentiation

<5-1> Construction of SAGE Library According to Each Stage of NK Cell Differentiation

Based on the results of SAGE in the Example 4, 4 different SAGE libraries were constructed according to each stage of NK cell differentiation (HSC, pNK, mNK(−OP9), mNK(+OP9)). From SAGE library of HSC, 19,830 unique transcripts were identified from total 44,998 tags, and among them, 12,899 specific genes were identified. From SAGE library of pNK, 17,745 unique transcripts were identified from total 40,771 tags, and among them, 11,684 specific genes were identified. Likewise, from SAGE library of mNK, 20,803 and 20,791 unique transcripts were each identified from 42,160 tags (mNK(−OP9)) and 42, 535 tags (mNK(+OP9)), and among them, 3,650 and 14,335 specific genes were identified respectively. On the whole, total 170,464 tags were identified from the above four SAGE libraries, from which 59,657 unique transcripts and 35,385 specific genes were identified. Among 59,657 unique transcripts, 77.9% were single copy, 16.8% showed 2-4 copies, 3.2% showed 5-9 copies, 1.9% had 10-99 copies, and just 0.2% had over 100 copies (Table 1).

TABLE 1
SAGE result according to each stage of NK cell differentiation

| Cells according | | | |
|---|---|---|---|
| to each stage | | Number of | |
| of | Number of | unique | Number of |
| differentiation | tags | transcripts | specific genes |
| HSC | 44,998 | 19,830 | 12,899 |
| pNK | 40,771 | 17,745 | 11,684 |
| mNK(−OP9) | 42,160 | 20,803 | 13,650 |
| mNK(+OP9) | 42,535 | 20,791 | 14,335 |
| Total | 170,464 | 59,657 | 35,385 |

The reflection of the expression patterns of genes known to have an effect on NK cell differentiation was also investigated based on the above result of SAGE. As a result, as expected, the numbers of mNK cell receptors such as granzyme (GenBank ID NM_{—}013542), NKG2A (GenBank ID AF106008), 2B4 (GenBank ID L19057), Ly49Q (GenBank ID AB033769) and CD94 (GenBank ID AF057714) were big in mNK cells but were not counted in HSC and pNK cells, either. IL-15 (GenBank ID U14332) was detected only in HSC and pNK cells. The expression of ID2 (GenBank ID BC006951) began from the stage of pNK cells (Table 2).

TABLE 2
SAGE result of differentiation related genes

| Gene | HSC | pNK | mNK(−OP9) | mNK(+OP9) |
|---|---|---|---|---|
| Granzyme | 0 | 0 | 508 | 664 |
| NKG2A | 0 | 0 | 6 | 3 |
| NK receptor | 1 | 0 | 17 | 17 |
| 2B4 | | | | |
| NK receptor | 0 | 1 | 2 | 6 |
| Ly-49Q | | | | |
| CD94 | 0 | 0 | 3 | 1 |
| IL-15 | 3 | 3 | 0 | 0 |
| Ly49G2 | 0 | 0 | 1 | 0 |
| ID-2 | 0 | 7 | 5 | 9 |

<5-2> Analysis of Differentiation Stage Specific Genes Expressed in Each NK Cell Differentiation Stage

It was reported that different genes were expressed according to NK cell differentiation stages, so that the present inventors identified differentiation stage specific genes. For the statistical significance, genes at least 4-fold counted were grouped and presented in a table.

As a result, 15 genes were confirmed to be highly expressed in HSC (Table 3). In particular, interleukin-1 receptor associated kinase (IRAK) involved in the NK cell activation and signal transduction. In consideration of the report that the ability to induce cytotoxicity in NK cell caused by IL-18 and the generation of IFN-γ by activated NK cell were seriously damaged and decreased in IRAK-deficient mouse, the analysis of the present invention was correctly done.

TABLE 3

| | | | | | MNK |
|---|---|---|---|---|---|
| Gene | GenBank ID | HSC | pNK | MNK (−OP9) | (+OP9) |
| Homeobox | AF15457 | 28 | 0 | 0 | 0 |
| protein MIX | | | | | |
| Pre-pro- | U97073 | 28 | 0 | 0 | 0 |
| proteinase 3 | | | | | |
| Myeloblastosis | M16499 | 11 | 1 | 0 | 1 |
| (Myb) | | | | | |
| oncogene | | | | | |
| Keratin | NM_010662 | 9 | 0 | 0 | 0 |
| complex 1, | | | | | |
| acidic, gene | | | | | |
| 13 | | | | | |
| PA- | AK002966 | 8 | 0 | 1 | 1 |
| phosphatase | | | | | |
| related | | | | | |
| phosphoesterase | | | | | |
| Interleukin | AK009132 | 7 | 0 | 0 | 0 |
| 1 receptor- | | | | | |
| associated | | | | | |
| kinase | | | | | |
| Gamma-parvin | BC011200 | 6 | 0 | 0 | 0 |
| Forkhead- | AF330105 | 4 | 1 | 1 | 0 |
| related | | | | | |
| transcription | | | | | |
| factor 1C | | | | | |
| RIKEN cDNA | AK017744 | 4 | 1 | 0 | 0 |
| 5730501N20 | | | | | |
| gene | | | | | |
| c-myc | X010223 | 4 | 0 | 0 | 1 |
| protein | | | | | |
| Ribosomal | AK002613 | 4 | 0 | 1 | 0 |
| protein L10A | | | | | |
| Oct 2b gene | X53654 | 4 | 0 | 0 | 0 |
| Microlite | AK015601 | 4 | 0 | 0 | 0 |
| Dihydrolipoamide | BC003368 | 4 | 0 | 0 | 0 |
| dihydrogenase | | | | | |
| Tracle | U81030 | 4 | 0 | 0 | 0 |

And, 30 other genes were exceptionally expressed in pNK cell stage (Table 4). Among them, c-kit ligand was confirmed to be essential for the complete differentiation into mNK cells and so the progress from premature NK cells into mature NK cells was inhibited in the absence of c-kit signal transduction. It was also reported that 2-microglobulin is involved in the beginning of the expression of Ly49 receptor and in the variety of NK cell receptors which are major regulators of NK cell differentiation. The expression of transformed Fc receptor affects the development and the function of NK cells, resulting in the decrease of the number of CD56+CD3− NK cells and further in cytopenia and other critical immunodeficiency syndroms. According to the result that genes known to regulate NK cell differentiation were expressed in the right stages as expected, the analysis of the present invention was correctly done.

TABLE 4

| | GenBank | | | MNK | MNK |
|---|---|---|---|---|---|
| Gene | ID | HSC | pNK | (−OP9) | (+OP9) |
| Lysozyme | BC002069 | 14 | 1321 | 2 | 3 |
| Ferritin H chain | BC012314 | 25 | 962 | 7 | 18 |
| Brevican | X87096 | 7 | 259 | 1 | 1 |
| Matrix | BC019135 | 0 | 69 | 0 | 0 |
| metalloproteinase | | | | | |
| 12 | | | | | |
| EIA-stimulated | AF084524 | 5 | 45 | 7 | 1 |
| gene cellular | | | | | |
| inhibitor | | | | | |
| c-kit ligand | M64262 | 0 | 62 | 0 | 0 |
| S100 calcium | BC027635 | 1 | 42 | 0 | 1 |
| binding protein | | | | | |
| A9 | | | | | |
| MPS1 protein | L20315 | 1 | 35 | 0 | 0 |
| Transglutaminase 2 | BC016492 | 0 | 25 | 1 | 1 |
| Serum and | AF139639 | 0 | 20 | 0 | 0 |
| glucocorticoid | | | | | |
| regulated protein | | | | | |
| kinase | | | | | |
| RIKEN cDNA | BC027496 | 0 | 18 | 0 | 0 |
| 5830413L19 | | | | | |
| Beta 2- | M10416 | 0 | 17 | 0 | 0 |
| microglobulin | | | | | |
| mRNA | | | | | |
| Interferon- | BC003804 | 0 | 17 | 0 | 0 |
| induced protein | | | | | |
| Milk fat globul | BC018577 | 3 | 16 | 0 | 1 |
| membrane protein | | | | | |
| EGF factor 8 | | | | | |
| Fc gamma receptor | M14215 | 3 | 15 | 1 | 1 |
| Cell-surface | U83172 | 0 | 13 | 0 | 1 |
| glycoprotein p91 | | | | | |
| Arginase 1 | BC050005 | 0 | 12 | 0 | 0 |
| Tumor Necrosis | M59378 | 1 | 12 | 0 | 2 |
| factor receptor 1 | | | | | |
| Retinoid-induced | AF330052 | 2 | 11 | 0 | 0 |
| serine | | | | | |
| carboxypeptidase | | | | | |
| Unidentified | BC023802 | 0 | 11 | 2 | 0 |
| protein FLJ11000 | | | | | |
| homologue | | | | | |
| Interleukin-18 | AF110803 | 0 | 10 | 0 | 0 |
| binding protein d | | | | | |
| precursor | | | | | |
| Chloride channel 7 | AK009435 | 0 | 9 | 1 | 0 |
| CD36 antigen | BC010262 | 0 | 8 | 0 | 0 |
| Zink finger | BC030186 | 1 | 8 | 1 | 0 |
| protein homologue | | | | | |
| Carbohydrate | J03723 | 0 | 7 | 3 | 0 |
| binding protein | | | | | |
| 35 | | | | | |
| C-type calcium | BC003218 | 0 | 7 | 0 | 0 |
| dependent | | | | | |
| carbohydrate | | | | | |
| Lipoprotein | NM_008509 | 0 | 7 | 0 | 0 |
| lipase | | | | | |
| v-maf lacertus | BC038256 | 0 | 6 | 0 | 0 |
| fibrosarcoma | | | | | |
| oncogene | | | | | |
| Interleukin 7 | NM_008372 | 0 | 5 | 0 | 0 |
| receptor | | | | | |
| Chemokine (C—C) | BC011092 | 0 | 5 | 0 | 0 |
| receptor 1 | | | | | |
| Neurophilline | AK002673 | 0 | 5 | 0 | 0 |
| (MGD\|MGI: 106206) | | | | | |

In the meantime, 27 genes were identified from mNK cell stage (Table 5). Among them, Src family tyrosin kinase ‘Fyn’ is known to be involved in the activation of NK cell.

TABLE 5

| | GenBank | | | MNK | MNK |
|---|---|---|---|---|---|
| Gene | ID | HSC | pNK | (−OP9) | (+OP9) |
| SERPINA3G | XM_127137 | 2 | 0 | 29 | 45 |
| GABA-A receptor | X51986 | 0 | 0 | 16 | 44 |
| subunit 6 | | | | | |
| LAPTm5 | U51239 | 5 | 4 | 18 | 25 |
| G-protein | BC049968 | 0 | 0 | 0 | 17 |
| signal | | | | | |
| regulator | | | | | |
| Decoy- | L41366 | 0 | 0 | 0 | 12 |
| stimulating | | | | | |
| factor GPI | | | | | |
| fixed mRNA | | | | | |
| Y box protein 3 | AK019465 | 0 | 0 | 10 | 17 |
| Osteopontin | J04806 | 0 | 1 | 2 | 14 |
| precursor | | | | | |
| Amyloid beta | AK021331 | 2 | 0 | 5 | 12 |
| (A4) precursor | | | | | |
| protein-binding | | | | | |
| family | | | | | |
| T cell receptor | U63547 | 0 | 0 | 8 | 11 |
| beta subunit | | | | | |
| analogue | | | | | |
| Immune related | BC005577 | 0 | 0 | 9 | 0 |
| nucleotide 1 | | | | | |
| Higher stage | NM_009480 | 0 | 1 | 0 | 8 |
| transcription | | | | | |
| factor 1 | | | | | |
| Olfactory | NM_146714 | 0 | 0 | 0 | 8 |
| receptor | | | | | |
| MOR267-7 | | | | | |
| Lymphocyte | M12056 | 0 | 0 | 7 | 1 |
| specific | | | | | |
| protein | | | | | |
| tyrosine kinase | | | | | |
| Osteoclast | AB013898 | 1 | 1 | 0 | 7 |
| cancer | | | | | |
| inhibitor | | | | | |
| Platelet active | BC024054 | 0 | 1 | 3 | 7 |
| receptor | | | | | |
| homologue | | | | | |
| Natural killer | AF016008 | 0 | 0 | 3 | 6 |
| cell protein 2- | | | | | |
| A1 | | | | | |
| Unidentified | BC023851 | 0 | 1 | 2 | 6 |
| protein | | | | | |
| MGC36662 | | | | | |
| Semaphorin 6A | AK004390 | 0 | 0 | 6 | 2 |
| precursor | | | | | |
| homologue | | | | | |
| Fyn proto- | BC032149 | 0 | 0 | 5 | 5 |
| oncogene | | | | | |
| Neurofilament | BC025872 | 0 | 0 | 2 | 5 |
| homologue, | | | | | |
| polypeptide | | | | | |
| Cornin | BC026634 | 1 | 1 | 6 | 2 |
| homologue, | | | | | |
| actin binding | | | | | |
| protein 2A | | | | | |
| Solute | BC015245 | 1 | 1 | 6 | 5 |
| transmitting | | | | | |
| family 6 | | | | | |
| Temporary | AK020001 | 0 | 0 | 5 | 4 |
| purine receptor | | | | | |
| P2Y10 homologue | | | | | |
| T cell receptor | X03802 | 0 | 1 | 5 | 4 |
| gamma chain | | | | | |
| Poly A | NM_011112 | 0 | 0 | 5 | 3 |
| polymerase | | | | | |
| alpha | | | | | |
| OPA-related | AK017825 | 0 | 0 | 5 | 1 |
| protein OIP5 | | | | | |
| analogue | | | | | |
| Mytogen | BC006708 | 1 | 0 | 5 | 4 |
| activated | | | | | |
| protein kinase | | | | | |
| 1 analogue | | | | | |

### EXAMPLE 6

### Investigation of Expression Patterns of Genes by RT-PCR

Semiquantitative RT-PCR was performed to investigate expression patterns of other genes, based on SAGE data. Primers for the RT-PCR were prepared according to target genes. All PCR mixtures were heated at 95° C. for 1 minute, and other PCR conditions were as follows; PCR with HSC and mNK cells was performed at 95° C. for 1 minute, at 55° C. for 1 minute and at 72° C. for 2 minutes, and PCR with premature NK cells was performed at 95° C. for 1 minute, at 60° C. for 1 minute and at 72° C. for 2 minutes, which were repeated 28 or 32 cycles, and then extension followed at 72° C. for 10 minutes. The amplified PCR products were electrophorezed and stained with ethidium bromide.

Gamma-parvin: SEQ. ID. No 3 and No 4,

Forkhead-related transcription factor 1c (Foxp1c): SEQ. ID. No 5 and No 6,

c-myc protein: SEQ. ID. No 7 and No 8,

Keratin complex (KC) 1: SEQ. ID. No 9 and No 10,

PA-phosphatase related phosphoesterase (PA-PRP): SEQ. ID. No 11 and No 12,

Interleukin 1 receptor-associated kinase (IRAK): SEQ. ID. No 13 and No 14,

Ribosomal protein L10A: SEQ. ID. No 15 and No 16,

Pre-pro-proteinase 3: SEQ. ID. No 17 and No 18,

Myeloblastosis oncogene: SEQ. ID. No 19 and No 20,

Carbohydrate binding protein (CBP) 35: SEQ. ID. No 21 and No 22,

IL-7 receptor: SEQ. ID. No 23 and No 24,

Lipoprotein lipase (LPL): SEQ. ID. No 25 and SEQ. ID. No 26,

Ferritin H chain: SEQ. ID. No 27 and No 28,

Matrix metalloproteinase (MMP) 12: SEQ. ID. No 29 and No 30,

Regulator of G-protein signaling (RGS): SEQ. ID. No 31 and No 32,

Serpina 3G: SEQ. ID. No 33 and No 34,

Purinergic receptor P2Y: SEQ. ID. No 35 and No 36,

Lymphocyte-specific protein tyrosin kinase (PTK): SEQ. ID. No 37 and No 38,

Semaphorin 6A precursor: SEQ. ID. No 39 and No 40,

CD122: SEQ. ID. No 41 and No 42,

Perforin: SEQ. ID. No 43 and No 44,

Beta-actin: SEQ. ID. No 45 and No 46

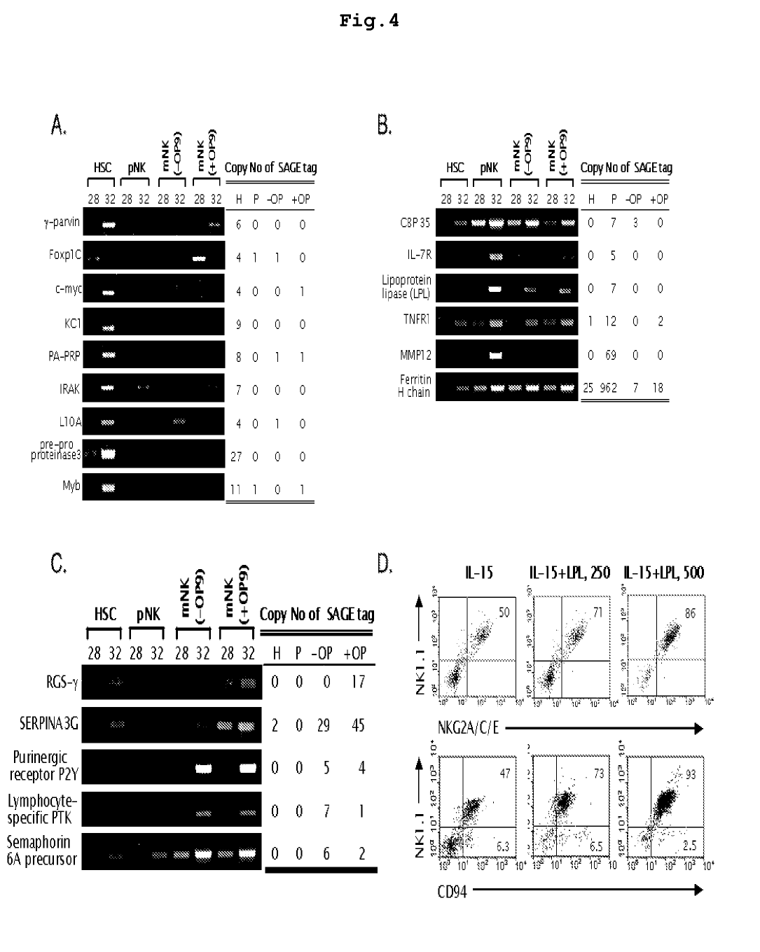

As a result, 9 genes, for example gamma-parvin, forkhead-related transcription factor 1c (Foxp1c), c-myc, pre-pro-proteinase 3, etc, were specifically expressed in HSC (FIG. 4a). IL-7R and matrix metalloproteinase 12 (MMP12) were exceptionally expressed in pNK cells (FIG. 4b). Purinergic receptor P2Y10 and lymphocyte-specific protein tyrosin kinase (PTK) were unusually expressed in mNK cells (FIG. 4c).

### EXAMPLE 7

### Effect of LPL on NK Cell Differentiation Stages

In the above Example 4, it was confirmed that lipoprotein lipase (referred as ‘LPL’ hereinafter) represented by SEQ. ID. No 47 was over-expressed in pNK cells during NK cell differentiation among many differentiation stage specific genes. LPL promotes NK cell proliferation but inhibits spontaneous cytotoxicity and activity of lymphokine-activated killer (LAK). In order to confirm whether pNK-specific expression of LPL was required for the differentiation into mNK cells, HSC cells were cultured for 6 days, which were then treated with IL-15 and LPL in the absence of OP9 stromal cells, followed by measuring the percentage of NK cells.

As a result, the NK cell percentage was increased more when HSC was treated with IL-15 and LPL together than when it was treated with IL-15 only (NK1.1+ NKG2A/C/E+ cell; 50% when it was treated with IL-15 only versus 71% and 86% each when treated with IL-15 and 250 ng/ml of LPL together and when treated with IL-15 and 500 ng/ml of LPL together) (FIG. 4d). The above results indicate that LPL plays an important role in the differentiation from pNK cells into mNK cells and the search of genes regulating NK cell differentiation was correctly done in the present invention.

### INDUSTRIAL APPLICABILITY

As explained hereinbefore, the method of the present invention for searching genes involved in the regulation of differentiation from stem cells into natural killer cells, in addition to SAGE, is very useful for identifying a novel gene having unfamiliar functions.

[Sequence List Text]

Nucleotide sequences represented by SEQ. ID. No 1 and No 2 are the primer sequences used for the PCR in the Example 3.

Nucleotide sequences represented by SEQ. ID. No 3 and No 4 are the primer sequences used for the RT-PCR with gamma-parvin in the Example 6.

Nucleotide sequences represented by SEQ. ID. No 5 and No 6 are the primer sequences used for the RT-PCR with forkhead-related transcription factor 1c in the Example 6.

Nucleotide sequences represented by SEQ. ID. No 7 and No 8 are the primer sequences used for the RT-PCR with c-myc protein in the Example 6.

Nucleotide sequences represented by SEQ. ID. No 9 and No 10 are the primer sequences used for the RT-PCR with keratin complex (KC) 1 in the Example 6.

Nucleotide sequences represented by SEQ. ID. No 11 and No 12 are the primer sequences used for the RT-PCR with PA-phosphatase related phosphoesterase (PA-PRP) in the Example 6.

Nucleotide sequences represented by SEQ. ID. No 13 and No 14 are the primer sequences used for the RT-PCR with interleukin 1 receptor-associated kinase (IRAK) in the Example 6.

Nucleotide sequences represented by SEQ. ID. No 15 and No 16 are the primer sequences used for the RT-PCR with ribosomal protein L10A in the Example 6.

Nucleotide sequences represented by SEQ. ID. No 17 and No 18 are the primer sequences used for the RT-PCR with pre-pro-proteinase 3 in the Example 6.

Nucleotide sequences represented by SEQ. ID. No 19 and No 20 are the primer sequences used for the RT-PCR with myeloblastosis oncogene in the Example 6.

Nucleotide sequences represented by SEQ. ID. No 21 and No 22 are the primer sequences used for the RT-PCR with carbohydrate binding protein (CBP) 35 in the Example 6.

Nucleotide sequences represented by SEQ. ID. No 23 and No 24 are the primer sequences used for the RT-PCR with IL-7 receptor in the Example 6.

Nucleotide sequences represented by SEQ. ID. No 25 and No 26 are the primer sequences used for the RT-PCR with lipoprotein lipase (LPL) in the Example 6.

Nucleotide sequences represented by SEQ. ID. No 27 and No 28 are the primer sequences used for the RT-PCR with ferritin H chain in the Example 6.

Nucleotide sequences represented by SEQ. ID. No 29 and No 30 are the primer sequences used for the RT-PCR with matrix metalloproteinase (MMP) 12 in the Example 6.

Nucleotide sequences represented by SEQ. ID. No 31 and No 32 are the primer sequences used for the RT-PCR with Regulator of G-protein signaling (RGS) in the Example 6.

Nucleotide sequences represented by SEQ. ID. No 33 and No 34 are the primer sequences used for the RT-PCR with serpina 36 in the Example 6.

Nucleotide sequences represented by SEQ. ID. No 35 and No 36 are the primer sequences used for the RT-PCR with purinergic receptor P2Y in the Example 6.

Nucleotide sequences represented by SEQ. ID. No 37 and No 38 are the primer sequences used for the RT-PCR with Lymphocyte-specific protein tyrosin kinase (PTK) in the Example 6.

Nucleotide sequences represented by SEQ. ID. No 39 and No 40 are the primer sequences used for the RT-PCR with semaphorin 6A precursor in the Example 6.

Nucleotide sequences represented by SEQ. ID. No 41 and No 42 are the primer sequences used for the RT-PCR with CD122 in the Example 6.

Nucleotide sequences represented by SEQ. ID. No 43 and No 44 are the primer sequences used for the RT-PCR with Perforin in the Example 6.

Nucleotide sequences represented by SEQ. ID. No 45 and No 46 are the primer sequences used for the RT-PCR with beta-actin in the Example 6.

Nucleotide sequence represented by SEQ. ID. No 47 is the nucleotide sequence of lipoprotein lipase.

Nucleotide sequence represented by SEQ. ID. No 48 is the amino acid sequence of a mouse protein.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1-7. (canceled)

8. A differentiation regulating agent, which regulates differentiation from stem cells into natural killer cells, containing one or more genes, as an effective ingredient, selected from a group consisting of homeobox protein MIX (AF15457), pre-pro-proteinase 3 (U97073), myeloblastosis (Myb) oncogene (M16499), keratin complex 1, acidic, gene 13 (NM_{—}010662), PA-phosphatase related phosphoesterase (AK002966), gamma-parvin (BC011200), forkhead-related transcription factor 1C (AF330105), RIKEN cDNA 5730501N20 gene (AK017744), c-myc protein (X010223), ribosomal protein L10A (AK002613), Oct 2b gene (X53654), microlite (AK015601), dihydrolipoamide dihydrogenase (BC003368), tracle (U81030), lysozyme (BC002069), ferritin H chain (BC012314), brevican (X87096), matrix metalloproteinase 12 (BC019135), EIA-stimulated gene cellular inhibitor (AF084524), S100 calcium binding protein A9 (BC027635), MPS1 protein (L20315), transglutaminase 2 (BC016492), serum and glucocorticoid regulated protein kinase (AF139639), RIKEN cDNA 5830413L19 (BC027496), interferon-induced protein (BC003804), milk fat globul membrane protein EGF factor 8 (BC018577), cell-surface glycoprotein p91 (U83172), arginase 1 (BC050005), tumor necrosis factor receptor 1 (M59378), retinoid-induced serine carboxypeptidase (AF330052), FLJ11000 homologue (BC023802), interleukin-18 binding protein d precursor (AF110803), chloride channel 7 (AK009435), CD36 antigen (BC010262), zink finger protein homologue (BC030186), carbohydrate binding protein 35 (J03723), C-type calcium dependent carbohydrate (BC003218), lipoprotein lipase (NM_{—}008509), v-maf lacertus fibrosarcoma oncogene (BC038256), interleukin 7 receptor (NM_{—}008372), chemokine (C-C) receptor 1 (BC011092), neurophilline (MGD|MGI:106206) (AK002673), SERPINA3G (XM_{—}127137), GABA-A receptor subunit 6 (X51986), LAPTm5 (U51239), G-protein signal regulator (BC049968), decoy-stimulating factor GPI fixed mRNA (L41366), Y box protein 3 (AK019465), osteopontin precursor (J04806), amyloid beta (A4) precursor protein-binding family (AK021331), T cell receptor beta subunit analogue (U63547), immune related nucleotide 1 (BC005577), higher stage transcription factor 1 (NM_{—}009480), olfactory receptor MOR267-7 (NM_{—}146714), lymphocyte specific protein tyrosine kinase (M12056), osteoclast cancer inhibitor (AB013898), platelet active receptor homologue (BC024054), natural killer cell protein 2-A1 (AF016008), unidentified protein MGC36662 (BC023851), semaphorin 6A precursor homologue (AK004390), neurofilament homologue polypeptide (BC025872), cornin homologue actin binding protein 2A (BC026634), solute transmitting family 6 (BC015245), temporary purine receptor P2Y10 homologue (AK020001), T cell receptor gamma chain (X03802), poly A polymerase alpha (NM_{—}011112), OPA-related protein OIP5 analogue (AK017825) and mytogen activated protein kinase 1 analogue (BC006708).

9. The differentiation-regulating agent as set forth in claim 8, wherein the differentiation-regulating agent is used for the treatment of cancer.

10. The differentiation regulating agent as set forth in claim 9, wherein the cancer is selected from a group consisting of breast cancer, melanoma and lung cancer.

11. A differentiation regulating agent, which regulates differentiation from stem cells into premature natural killer cells, containing one or more genes, as an effective ingredient, selected from a group consisting of homeobox protein MIX (AF15457), pre-pro-proteinase 3 (U97073), myeloblastosis (Myb) oncogene (M16499), keratin complex 1, acidic, gene 13 (NM_{—}010662), PA-phosphatase related phosphoesterase (AK002966), gamma-parvin (BC011200), forkhead-related transcription factor IC (AF330105), RIKEN cDNA 5730501N20 gene (AK017744), c-myc protein (X010223), ribosomal protein L10A (AK002613), Oct 2b gene (X53654), microlite (AK015601), dihydrolipoamide dihydrogenase (BC003368) and tracle (U81030).

12. The differentiation-regulating agent as set forth in claim 11, wherein the differentiation-regulating agent is used for the treatment of cancer.

13. The differentiation regulating agent as set forth in claim 12, wherein the cancer is selected from a group consisting of breast cancer, melanoma and lung cancer.

14. A differentiation regulating agent, which regulates differentiation from premature natural killer cells into mature natural killer cells containing one or more genes, as an effective ingredient, selected from a group consisting of lysozyme (BC002069), ferritin H chain (BC012314), brevican (X87096), matrix metalloproteinase 12 (BC019135), EIA-stimulated gene cellular inhibitor (AF084524), S100 calcium binding protein A9 (BC027635), MPS1 protein (L20315), transglutaminase 2 (BC016492), serum and glucocorticoid regulated protein kinase (AF139639), RIKEN cDNA 5830413L19 (BC027496), interferon-induced protein (BC003804), milk fat globul membrane protein EGF factor 8 (BC018577), cell-surface glycoprotein p91 (U83172), arginase 1 (BC050005), tumor necrosis factor receptor 1 (M59378), retinoid-induced serine carboxypeptidase (AF330052), FLJ11000 homologue (BC023802), interleukin-18 binding protein d precursor (AF110803), chloride channel 7 (AK009435), CD36 antigen (BC010262), zink finger protein homologue (BC030186), carbohydrate binding protein 35 (J03723), C-type calcium dependent carbohydrate (BC003218), lipoprotein lipase (NM_{—}008509), v-maf lacertus fibrosarcoma oncogene (BC038256), interleukin 7 receptor (NM_{—}008372), chemokine (C-C) receptor 1 (BC011092) and neurophilline (MGD|MGI: 106206).

15. The differentiation-regulating agent as set forth in claim 14, wherein the differentiation-regulating agent is used for the treatment of cancer.

16. The differentiation regulating agent as set forth in claim 15, wherein the cancer is selected from a group consisting of breast cancer, melanoma and lung cancer.

17. A differentiation regulating agent, which regulates differentiation of mature natural killer cells, containing one or more genes, as an effective ingredient, selected from a group consisting of SERPINA3G (XM_{—}127137), GABA-A receptor subunit 6 (X51986), LAPTm5 (U51239), G-protein signal regulator (BC049968), decoy-stimulating factor GPI fixed mRNA (L41366), Y box protein 3 (AK019465), osteopontin precursor (J04806), amyloid beta (A4) precursor protein-binding family (AK021331), T cell receptor beta subunit analogue (U63547), immune related nucleotide 1 (BC005577), higher stage transcription factor 1 (NM_{—}009480), olfactory receptor MOR267-7 (NM_{—}146714), lymphocyte specific protein tyrosine kinase (M12056), osteoclast cancer inhibitor (AB013898), platelet active receptor homologue (BC024054), natural killer cell protein 2-A1 (AF016008), unidentified protein MGC36662 (BC023851), semaphorin 6A precursor homologue (AK004390), neurofilament homologue polypeptide (BC025872), cornin homologue actin binding protein 2A (BC026634), solute transmitting family 6 (BC015245), temporary purine receptor P2Y10 homologue (AK020001), T cell receptor gamma chain (X03802), poly A polymerase alpha (NM_{—}011112), OPA-related protein OIP5 analogue (AK017825) and mytogen activated protein kinase 1 analogue (BC006708).

18. The differentiation-regulating agent as set forth in claim 17, wherein the differentiation-regulating agent is used for the treatment of cancer.

19. The differentiation regulating agent as set forth in claim 18, wherein the cancer is selected from a group consisting of breast cancer, melanoma and lung cancer.

20. A screening method for a gene regulating the differentiation from stem cells into natural killer cells comprising the following steps:
1) Synthesizing cDNA after separating whole RNA from stem cells or cells from stage of cell differentiation;
2) Separating tag after digesting the cDNA of the step 1;
3) Connecting each tag separated in the step 2 and then analyzing nucleotide sequence thereof; and
4) Quantifying the expression of the gene, based on the analyzed nucleotide sequence above, by using SAGE (Serial Analysis of Gene expression) analyzing program.

21. A screening method for a gene regulating the differentiation from stem cells into natural killer cells as set forth in claim 20, which further comprises clustering SAGE data obtained from the step 4 by computer clustering program with expression pattern.

22. A screening method for a gene regulating the differentiation from stem cells into natural killer cells as set forth in claim 20, wherein the stem cells or cells from stage of cell differentiation are HSCs(haematopoietic stem cell), pNK cells(precursor natural killer cells) and mNK cells(mature natural killer cells).
